(19) United States
(12) Patent Application Publication
(10) Pub. No.: US 2008/0301977 A1
(43) Pub. Date: Dec. 11, 2008

(54) TOE STRAP PROTECTOR, AND FOOTWEAR COMPRISING SAID PROTECTOR

(76) Inventors: Andrew Roberts, Woodley (GB); Encarna Perez, Dusseldorf (DE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/930,256

(22) Filed: Oct. 31, 2007

(30) Foreign Application Priority Data
Nov. 7, 2006 (EP) 06023092.7

(51) Int. Cl. A43B 7/26; A61F 13/06

(52) U.S. Cl. 36/94; 128/893

(57) ABSTRACT

The present invention relates to a protector for a toe strap of a footwear such, in particular in the form of a shoe, a sandal or a flip-flop, comprising a tube or a tube-like body with a first end and an opposite second end along its longitudinal axis, in particular in the form of a circumferential sleeve, which at least partially encloses a space, with a first opening at the first end and an opposite second opening at the second end for the at least partial inclusion of a toe strap, wherein an inner wall of the tube or tube-like body extends from the first to the second end and wherein an outer wall extends from the first to the second end, wherein the tube or tube-like body, in particular the circumferential sleeve, comprises a slit extending from the first end to the second end and from the outer to the inner wall so that the tube or tube-like body, in particular the circumferential sleeve, comprises adjacent longitudinal first and second areas, in particular contact areas which are facing each other and wherein the tube or tube-like body comprises an elastomeric material.

Furthermore, the present invention relates to a footwear comprising said protector according to the present invention.

The present invention is concerned with a protector for a toe strap of a footwear such as a shoe, sandal, flip-flop or the like as well as with a footwear such as a shoe, sandal and flip-flop comprising said protector.

Today's footwear is available in a huge variety of different forms and types which accommodate the specific needs of the individual user. However, being a mass product it is sometimes difficult to fulfill the needs of each wearer. Therefore the use of insoles or other types of prosthesis can sometimes not be avoided. This holds true for everyday's footwear and sports footwear as well as for sandals.

In DE 44 42 735 A1 an insole is described which is provided with means for adjusting and maintaining the distance between adjacent toes. This means is reported to be in the form of a plate or a pin and can be made from metal or plastics. A pin which is used in a shoe for the adjustment of the toe is also described in JP 10233401 A and JP 11169201 A.

A shoe being adapted for orthopedic uses is disclosed in DE 201 16 526 U1, in particular for the therapy of Hallux-Valgus. In the area of the toes the position of a pin protruding upwardly from the insole can be adjusted depending on the needs of the user. Such a pin can be made from rubber or can be at least partially covered by a rubber layer.

DE 20 2004 006 639 U1 is aiming at protecting the area between adjacent toes, in particular between the big toe and the first finger, when wearing sandals or flip-flops. The device of DE 20 2004 006 639 U1 comprises two interconnected halves which are forming a V-like angle. Both halves have at least partially the form of outwardly curved half-tubes in order to accommodate the anatomy of adjacent interior toe sections when being worn. The protector device of DE 20 2004 006 639 U1 has to be inserted first in the intermediate space between adjacent toes before the foot can be placed in a sandal. The toe strap of said sandal has to be inserted between the outer surfaces of the interconnected halves of the toe protector. Sometimes the use of said protector can be problematic since the half-tube like shape of one or both halves does not properly accommodate to the anatomy of the toes of the wearer thereby giving either a loose fit or a tight, discomfortable sensation. Moreover, when the toe strap of the sandal happens to slip out of the V-shaped slit formed by both halves of the protector it can not be easily inserted again. Naturally, the section of the protector where both halves are joined experiences large forces during use and is therefore easily damaged or even completely cut.

According to German utility model G 93 03 990.5 the wearing comfort of a sandal can be improved if in addition to the toe strap a transversal rib is provided which extends from said strap and which can be placed between the toes and the pad of the foot.

Instead of providing a shoe as such with a specific means to alleviate the wearing comfort DE 600 02 326 T2 discloses the use of a sleeve for the protection of a toe which is made from a fabric comprising thermoplastic fibers and a layer of a viscoelastic gel having been applied to said fabric. In this manner, a rather robust protective means is provided which nevertheless has a low thickness and a soft-touch feeling.

It has been desirable to provide footwear, in particular light footwear such as a sandal or a flip-flop, which can be safely and reliably worn.

Therefore, the object underlying the present invention has been to provide a means to improve the wearing comfort of a shoe, sandal, flip-flop or the like, in particular without the need to take care of individual anatomical preferences of the wearer.

The problem underlying the present invention has been solved by a protector for a toe strap of a footwear, in particular in the form of a shoe, sandal or flip-flop comprising a tube or a tube-like body with a first end and an opposite second end along its longitudinal axis, in particular in the form of a circumferential sleeve, which at least partially encloses a space, with a first opening at the first end and an opposite second opening at the second end for the at least partial inclusion of a toe strap, wherein an inner wall of the tube or tube-like body extends from the first to the second end and wherein an outer wall extends from the first to the second end, wherein the tube or tube-like body, in particular the circumferential sleeve, comprises a slit extending from the first end to the second end and from the outer to the inner wall so that the tube or tube-like body, in particular the circumferential sleeve, comprises adjacent longitudinal first and second areas, in particular contact areas, which are facing each other and wherein the tube or tube-like body comprises an elastomeric material.

A toe strap in the meaning of the present invention can be a more flexible or a more rigid toe strap, e.g. made from textiles, polymeric materials, strings, fabrics or the like, or can even be a pin.

The tube or tube-like body is leaving an inner space which can engage at least part of a toe strap. In particular due to the elastic characteristics of the tube or tube-like body the diameter of this inner space in a transverse direction perpendicular to the longitudinal axis of the tube or tube-like body can be easily adjusted to the size or the width of a toe strap.

According to another embodiment the longitudinal axis of the tube or tube-like body is placeable in the space between the first and second opening without touching the inner wall.

In one embodiment the tube or tube-like body has an essentially circular cross-section at least along the major portion of its longitudinal axis.

In a preferred embodiment the tube or tube-like body at least along a major portion of its longitudinal axis has a non-circular cross-section with a major transverse axis lying in said cross-section combining those opposite portions of the tube or tube-like body which are furthest apart from each other, and a minor transverse axis being perpendicular to said major transverse axis and crossing said axis at half of its length. In this manner four quadrants are generated.

Thus, there are two adjacent quadrants on one side of the major transverse axis which are facing to a first toe and there are the two remaining adjacent quadrants on the other side of the major transverse axis which are facing to the adjacent second toe. Among these two pairs of quadrants there are two adjacent quadrants on one side of the minor transverse axis which are adapted to face into the front direction of a footwear when the protector is placed on the toe strap of such footwear, and there is another pair of adjacent quadrants lying on the other side of the minor transverse axis which are adapted to direct into the heel direction of the footwear.

In a preferred embodiment the major transverse axis has a length in the range from about 5 mm to about 25 mm, in particular from about 6 mm to about 20 mm, e.g. from 8 mm to 18 mm or from 9 mm to 16 mm, and/or the minor transverse axis has a length from about 3 mm to about 20 mm, in particular from about 4 mm to about 15 mm, e.g. from 5 mm to 14 mm.

In addition, the average thickness of the tube or tube-like body is usually in the range of about 1 mm to about 5 mm, in particular from about 1.5 mm to 4 mm. The hole or space generated by the tube or tube-like body preferably has an average width or length in the cross-sectional plane, i.e. a distance between opposite inner wall setions, in the range from about 3 mm to about 20 mm, in particular from about 4 mm to about 15 mm, e.g. 8 mm or 10 mm. If the cross-sectional shape of the hole or space is not symmetrical, e.g. oval or elliptical, the width in the direction of the major transverse axis is preferably in the range from about 5 mm to about 20 mm, in particular from about 6 mm to about 15 mm, e.g. 8 mm, and the width in the direction of the minor transverse axis preferably is in the range from about 2 mm to about 12 mm, in particular from about 3 mm to about 8 mm, e.g. 5 mm.

In general, the slit can be located at any position, e.g. on or off the major or minor transverse axis. In a preferred embodiment the slit is located in one of the quadrants. That is, in this embodiment the slit is not lying on a position where the major or minor transverse axes cross the tube or tube-like body, the major and minor axis lying in the cross-sectional plane. For example, the slit can be located at an angle of at least more than 5° off of the major transverse axis, in particular more than 15° off of said major axis, and/or at an angle of at least more than 5° off of the minor transverse axis, in particular more than 15° off of said minor axis.

In particular, the slit is located at an angle from 0° to 50°, preferably from 0° to 45°, off of the major transverse axis. According to a preferred embodiment the slit is located at an angle of at least more than 25° off of the major transverse axis and at an angle of at least more than 40° off of the minor transverse axis. In a particular preferred embodiment the slit is located at an angle of about 15° to about 40°, for example from 25° to 35° e.g. 30°, off of the major transverse axis. In the aforementioned embodiments the slit most preferably is located in one of those two adjacent quadrants which are designed to direct into the heel direction of footwear.

Usually, in one embodiment the slit is essentially parallel to the longitudinal axis of the tube or tube-like body.

Alternatively, the slit can also be bent between the first end and the second end. In this case, it is preferred when the longitudinal curvature of the slit comprises one point of inflection.

Owing to the anatomy of the foot, in particular of the toes, the width of the tube or tube-like body, when placed on a toe strap between adjacent toes, is usually larger in the front to heel direction than the width perpendicular thereto, that is between adjacent toes. In principle, the tube or tube-like body of the protector of the present invention can exhibit over a part of or over the entire longitudinal length a regular or irregular non-circular cross-sectional shape. Among these cross-sectional shapes the oval, elliptic or trapezoidal or oval-like, elliptic-like or trapezoidal-like shapes are preferred. It is also possible to provide the tube or tube-like body partially or completely with a rectangular, square-like, triangular or any other polygonal cross-sectional shape.

Preferably, with protectors of the present invention the distance between opposite sections of the outer wall which are lying on a transverse axis perpendicular to said longitudinal axis which is located at or adjacent to the first (top) end and/or the second (bottom) end is larger than the distance between opposite sections of the outer wall which are lying on a transverse axis perpendicular to said longitudinal axis which is located between the first end and the second end.

Accordingly, in another preferred embodiment the longitudinal cross-section of the tube-like body provides an outer wall which is at least over a section of the circumferential angle or essentially circumferentially essentially concavely curved.

Alternatively, it is preferred that the longitudinal cross-section of the tube or tube-like body provides an outer wall which at least over a section of its circumferential angle or essentially circumferentially has at least partially a curvature which essentially has the shape of a circle segment. It is preferred that the radius of the circle of said circle segment is in the range of about 15 mm to about 80 mm, in particular from about 20 mm to about 70 mm.

In one embodiment the longitudinal first and second contact areas have an average distance of no more than 5 mm, in particular of no more than 2 mm, in the ground state of the tube or tube-like body. In another embodiment according to the invention the first and second contact areas abut each other at least partially in the ground state of the tube or tube-like body. The ground state of the tube or tube-like body in the meaning of the present invention is a configuration adopted by said tube or tube-like body when no external or internal forces are applied thereto.

In general, it is preferred if the slit is rather narrow so that the first and second contact areas are either in close contact or are not far apart from each other in the ground state.

In this regard, it is also possible that the first and second contact areas abut each other at least partially in the ground state of the tube or tube-like body.

Usually, the wearing comfort can be increased if when the first and second contact areas abut each other the outer wall at said abutting section of the slit is essentially continuous.

The protector of the present invention further provides for an embodiment wherein the distance between the inner and the outer wall of the tube or tube-like body measured along the first and/or second contact areas is larger than the shortest distance between the inner and the outer wall of the tube or tube-like body measured adjacent to the first and second contact areas.

In another aspect of the present invention the first contact area is provided with a male closure device and wherein the second contact area is provided with a female closure device wherein the male and female closure devices are adapted to releasably engage each other. By using corresponding closure devices an even tighter and more secure fit of the protector when placed on a toe strap can be obtained.

It has been found to be advantageous to construe the protector of the present invention at least partially in a symmetrical manner. For example, it is preferred to construe the tube or tube-like body in such a manner that it is essentially symmetric to a mirror plane lying in the plane of the longitudinal cross-section which is oriented from the front to the heel section, and/or to construe the upper and lower parts or halves of the tube or tube-like body in such a way that they are symmetric in relation to a mirror plane lying in the plane of the transverse cross-section generated at about half the length of the longitudinal axis of said tube or tube-like body. In such a manner the usage of the protector of the present invention is facilitated in that it can be exchangeably placed on the left and the right shoe, sandal or flip-flop. Moreover, it can be worn with the very same comfort when placed upside down. Therefore, according to another preferred embodiment the first end and/or the second end of the tube or tube-like body is/are essentially flat, in particular parallel to each other.

Due to the elastic characteristics of the tube or tube-like body the space within the tube or tube-like body does not necessarily have to have the dimensions of the toe strap, but can be smaller or bigger since the elastomeric material is both compressible and extendable. In particular due to the slit also very large toe strap dimensions can be accommodated by the protector of the present invention.

According to another embodiment the inner wall and/or the outer wall has/have at least partially or essentially completely a smooth or an irregular surface. For example, instead of being smooth the inner and/or the outer wall may exhibit at least partially a pattern of ridges is and/or grooves, for example oriented in the plane of the cross-section of the tube or tube-like body or perpendicular to said plane. By use of an irregular surface on the inner wall the grip of the protector on the toe strap can be enhanced thereby decreasing the risk that the protector might easily move up and down the toe strap. To apply an irregular surface pattern to the outer wall, in particular in those regions which will be in contact with the toes when being worn, can enhance the wearing comfort as well as assist in keeping the protector in place during use.

Preferably, the elastomeric material used for the manufacture of the protector of the present invention is a natural or synthetic rubber or a thermoplastic elastomer (TPE). More preferably, the elastomeric material is a silicone rubber, in particular a liquid silicone rubber.

Suitable synthetic elastomeric polymeric materials comprise for example polymers made from conjugated dienes, for example, isoprene, butadiene, or chlorobutadiene, as well as from copolymeric materials made from conjugated dienes and vinyl derivatives such as styrene and acrylonitrile. Exemplarily, suitable synthetic rubber materials comprise isoprene rubber, butadiene rubber, chloroprene rubber, styrene butadiene rubber (SBR), nitrilo-butadiene rubber (NBR), also in hydrogenated form, ethylene-propylene-(diene) rubber (EPM, EPBM), ethylene vinyl acetate rubber, silicone rubber also including liquid silicone rubber.

Suitable liquid silicones are usually based on polyorganosiloxanes. These polymers are provided with siloxane chains (—Si—O—Si—O) in which the free valances of the Si atoms are saturated by use of hydrogen or an organic group such as methyl, phenyl, vinyl, or by use of hydroxyl groups. In general, liquid silicones can comprise around 1,000 repeating units. In contrast, solid silicone rubbers comprise around 6,000 to 10,000 repeating units. The vulcanization reaction usually takes place between a silane group (Si—H) and a vinyl group by way of a transition metal catalyzed hydrosilylation. Liquid silicones usually are based on a two-component mixture comprising vinylpolysiloxanes having a low viscosity on the one hand, and hydrogen functionalized polysiloxanes or silanes on the other hand. Suitable catalysts are based on noble metals, in particular platinum. In general, the amount of catalyst needed is in the ppm range. By way of addition reaction which usually does not furnish any side products vinyl functionalized polysiloxanes are bonded to hydrogen functionalized siloxanes. Preferably, in the two-component system it is the vinyl functionalized polysiloxane which already comprises the catalyst. Particularly suitable hydrogen functionalized siloxanes or silanes comprise methyl hydrogen siloxane, preferably comprising at least three SiH groups in one molecule. Suitable liquid silicones or their respective two-component systems are well known to the person skilled in the art, and can, for example be purchased from GE Bayer Silicones under the tradename Siloprene® LSR; from the company Wacker Chemie under the tradename Elastosil® LR, from the company Dow Corning under the tradename C6-550 or Silastic®, or from the company Rhodia under the tradename Silbione®. Of course, different silicones, e.g. different types of silicones can also be employed in admixture.

The vulcanization velocity can be controlled via the catalyst concentration and/or by adding an inhibitor. Suitable inhibitors comprise higher alcohols such as n-butanol, n-pentanol, n-hexanol, and preferably 1-ethinyl-1-cyclohexanol.

The tube or tube-like body, for example, is made by injection molding, cold polymerization, extrusion, casting, compression molding or foaming techniques. In particular the tube or tube like body is made by an injection molding process, preferably in one piece.

Suitable injection molding machines for the injection molding of liquid silicones are known to the person skilled in the art, and can for example be derived from DE 40 14 244 A1, DE 101 45 160 A1 or DE 103 027 A1. The vulcanization temperature usually is in the range from 130 to 230° C., in particular in the range from 140 to 220° C., and most preferred from 145 to 170° C. For example, according to DE 40 14 244 A1 the reactive two-component system can be jointly transferred into the mold by use of a cooled conduit. It is of course also possible to conduct the curing of liquid silicones at rather low temperatures (cold polymerization, e.g. 40° C.) thereby regularly accepting longer reaction times, e.g. 24 h, and also cycle times in order to obtain an even softer product, or products with heavily curved geometries.

Suitable elastomeric materials also comprise thermoplastic elastomers. These thermoplastic elastomers usually represent graft polymers and copolymers having thermoplastic sequences and elastomeric sequences which are essentially incompatible with each other. The soft elastomeric sequences usually have a glass transition temperature which is below ambient temperature whereas the harder thermoplastic sequences regularly have a glass transition temperature which is above ambient temperature. Thermoplastic elastomers comprise for example amide elastomers, copolyester elastomers, polyolefin elastomers, polystyrene elastomers, and polyurethane elastomers. Thermoplastic polyamide elastomers usually are made of block copolymers having hard polyamide sequences and soft polyether and/or polyester sequences. Thermoplastic copolyester elastomers are usually based on polyalkylene ether diol sequences (and/or long chained aliphatic dicarbonic esters) and sequences of partially crystalline polybutulene terephthalate. Suitable polyolefin elastomers are generally based on mixtures of isotactic polypropylene and ethylene-propylene rubbers. Thermoplastic polystyrene elastomers (TPS) are usually prepared via anionic polymerization and comprise sequences made from styrene on the one hand and butadiene on the other hand (SB, SBS). Thermoplastic polyurethane elastomers are usually made via a polyaddition reaction of long chained diols, such as 1,4-butane diol, 1,6-hexane diol, polyether diols, polyester diols or polycarbonate diols, with aliphatic diisocyanates such as HDI or IPDI. The aforementioned elastomeric materials as well as their preparation are known to a person skilled in the art.

According to an alternative embodiment of a protector of the present invention the tube or tube-like body is comprised of an inner wall segment and an outer wall segment. The inner wall segment of the tube or tube-like body in this embodiment at least partially is made from a polymeric material being more rigid, in particular a thermoplastic polymer, than the material of the outer wall segment of the tube or tube-like body, in particular a silicone elastomer or a thermoplastic elastomer (TPE). The inner wall segment and the outer wall segment in this embodiment are forming the tube or tube-like body. That is, the inner wall segment has a dimension in the direction of the outer wall and the outer wall segment has a dimension in the direction of the inner wall segment. In another embodiment one or two or more layers can be incorporated between the inner wall segment and the outer wall segment.

Typical thermoplastic polymers which can be used for the inner wall segment are polyolefins, such as polyethylene and polypropylene and their copolymeric forms, polyvinylchloride, polysulfones, polyetherketones, polyesters such as polybutyleneterephthalate and polyethyleneterephthalate, polycycloolefins, polyacrylates, polymethacrylates, polyamides, polycarbonates, polyurethanes, polyacetals, e.g. POM, polystyrene, styrene copolymers such as ABS, ASA and SAN, and polyphenyleneyether.

For the outer wall segment those elastomeric materials and preferred elastomeric materials can be used which already have been described above for the tube or tube-like body. Preferably, with the aforementioned embodiment said tube or tube-like body is made via a 2K injection molding process.

2K injection molding processes are well known in the art and are for example described in U.S. Pat. No. 3,479,425.

The protector for a toe strap of the present invention as described above can be used and is adapted for the use with footwear such as a shoe, a sandal or a flip-flop being provided with at least one toe strap. Due to the elastic characteristics and the slit of the protector device such a toe strap can be easily and reliably engaged and is safely held in place during use. Accordingly, with the present invention also footwear, in particular a shoe, a sandal or a flip-flop comprising at least one protector for a toe strap according to the present invention is claimed. In this context, it is also possible that the protector is an integral part of the insole and/or the strap of the footwear.

Most preferably, when worn with footwear the slit of the tube or tube-like body of the protector is directing towards the front and in particular the heel region of said footwear. In another preferred embodiment, the slit is directing in an off-center manner towards the front region and in particular the heel region of the footwear.

With the present invention it has been found that a protector for a toe strap of a footwear such as a shoe, a sandal or a flip-flop is provided, which allows for a save and reliable usage, which does not easily slip off a toe strap and which enhances the wearing comfort of a footwear being provided with a toe strap significantly. The rubbing and pain caused by toe straps can efficiently be reduced or even eliminated. Moreover, not only that the protector device of the present invention can be used exchangeably for toe straps of footwear of the right and the left foot, the very same protector device of the present invention which is used as for a small sized footwear can as well be used for large sized footwear. Furthermore, the aforementioned beneficial results can be obtained irrespective of the size and shape of the toe strap. Thus, the protector for a toe strap according to the present invention can cope with a variety of different types and sizes of footwear as well as toe straps.

Further characteristics and advantages of the invention will be more apparent from the detailed description of a preferred, non-exclusive embodiment of a protector according to the invention, which is described as a non-limiting example with the annexed drawings, in which

FIG. 1 shows a schematic perspective top view of a section of a first embodiment of a protector according to the present invention;

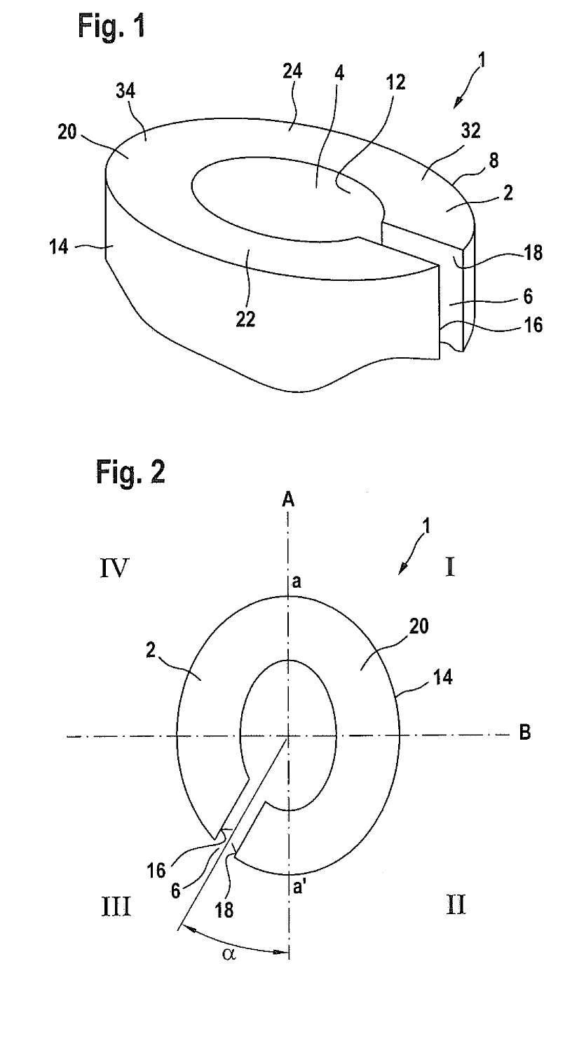

FIG. 2 shows a top view of the embodiment of FIG. 1;

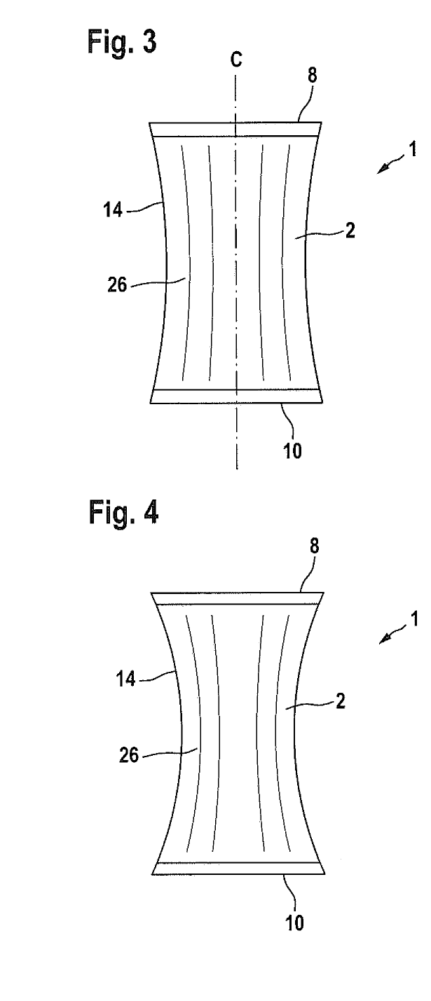

FIG. 3 shows a schematic side view of a first protector device of the present invention;

FIG. 4 shows a schematic side view of a second embodiment of a protector according to the present invention;

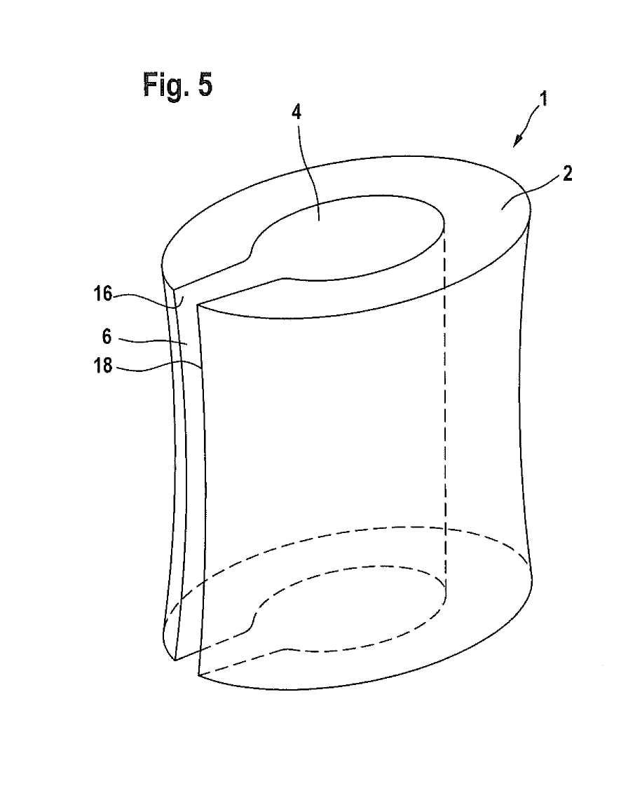

FIG. 5 shows a schematic perspective top view of a third embodiment of a protector according to the present invention;

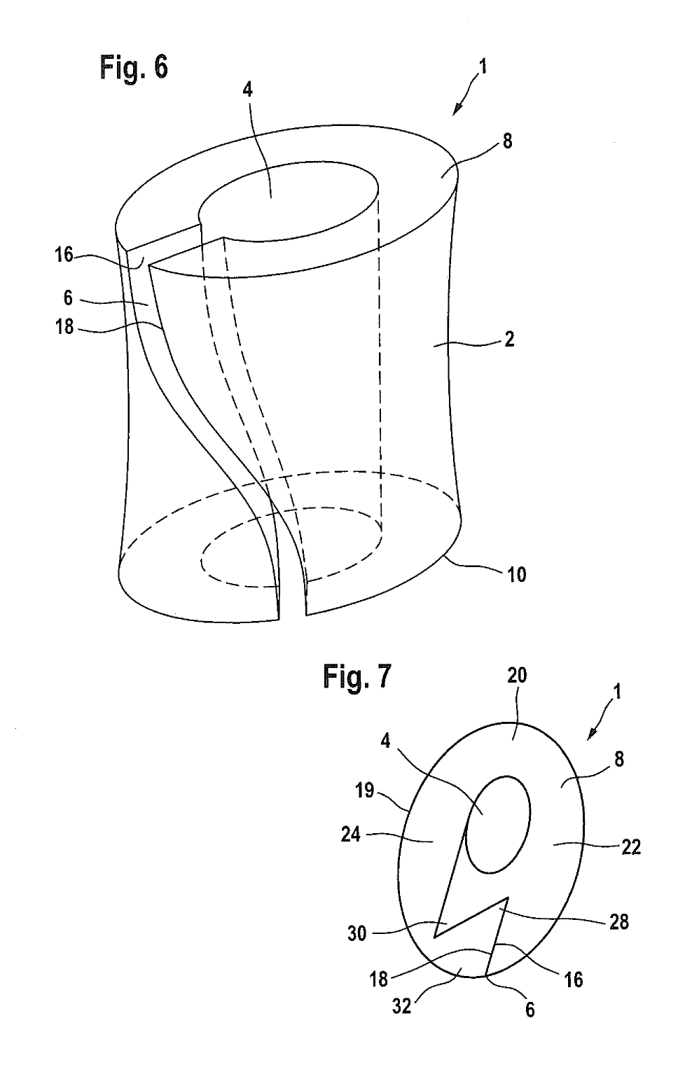

FIG. 6 shows a schematic perspective top view of a fourth embodiment of a protector according to the present invention; and

FIG. 7 shows a schematic top view of a fifth embodiment of a protector according to the present invention.

In FIG. 1 a top section of a protector for a toe strap, in the following also just referred to as protector 1, is depicted. The tube-like body 2 leaves an inner space 4 for the inclusion of a toe strap (not shown). The tube-like body 2 is provided with a slit 6 which extends from the first end 8 at the top to the second end 10 at the bottom (not shown) of protector 1 as well as from the inner wall 12 to the outer wall 14 of the tube-like body 2 thereby creating an opening through which a toe strap can be introduced into the inner space 4. Due to the elastomeric material on which the tube-like body 2 is based the slit 6 can be easily opened up and will fold back once the toe strap is inserted because of its resilient nature. The protector 1 is depicted in FIG. 1 in a ground state in which the opposite first and second contact areas 16 and 18 created by the slit 6 are not abutting each other but are keeping a slight distance of, for example, 0.1 mm to 2.5 mm, e.g. 1.5 mm. The tube-like body 2 has a longitudinal dimension and a transversal dimension and is essentially symmetrical upon its longitudinal cross-sectional mirror plane (apart from FIG. 6). According to the embodiment shown in FIG. 1 the thickness of the wall or sleeve 34 of the tube-like body 2 can be thinner in the front transverse section 20 than in the opposing longitudinal sections 22, 24 of said wall. Whereas the front transverse section 20 may have a thickness of about 2 mm the longitudinal sections 22, 24 of the wall may have an average thickness of about 3.5 mm. Usually, the section 32 of the protector 1 which comprises the slit 6 is directing towards the heel whereas the opposite transverse front section 20 is directing to the front part of a footwear when placed on a toe strap of, for example, a flip-flop. As can be derived from FIG. 1 the slit 6 is located in an off center manner which will be described in more detail with respect to FIG. 2.

FIG. 2 shows a schematic top view of the protector 1 of FIG. 1. The protector 1 is divided into four quadrants I to IV by a coordinate system generated by a major transverse axis A and a minor transverse axis B. The major transverse axis A lies essentially in the longitudinal front to heel cross-sectional mirror plane of the protector 1. It connects those portions of the outer wall 14 of the tube-like body 2 which are furthest apart from each other. The minor transverse axis B is perpendicular to the major transverse axis A and intersects the major transverse axis A at half the distance between points a and a′ on the outer wall 14. As can be easily derived from FIG. 2 the embodiment of a protector 1 has a slightly tapered transverse cross-section, that is at least some points on the outer wall 14, at least on the top 8, in quadrants I and IV are further apart from each other than at least some points in quadrants II and III.

Irrespective of the outer shape of the protector I the inner space 4 is apart from the slit 6 usually designed rather symmetrically.

Whereas regularly the broader part of the protector 1 including the front transverse section 20 of the tube-like body 2 is directing towards the front of a footwear the more narrow part and/or that part containing the slit 6 is directing towards the heel region of a footwear. Preferably, as shown in FIG. 2, the slit 6 is located in quadrant III (or II) and is not placed on the major transverse axis A or on the minor transverse axis B. In a preferred embodiment, the angle generated between the slit 6, or its first and second contact areas 16 and 18, and the major transverse axis A lies in the range from about 20° to 40°, in particular, as shown in FIG. 2, at about 30°. Alternatively, in another embodiment the slit 6 could also be placed on the tube-like body in one of quadrants I or IV (as long as not lying on the major transverse axis A or the minor transverse axis B).

In FIG. 3 a schematic side view of the protector 1 according to FIGS. 1 and 2 is shown. The diameter at the first (top) end 8 and the second (bottom) end 10 is larger than in the section between these first and second ends. As depicted in FIG. 3 the outer wall 14 may have the form of a circle segment, the underlying circle having, for example, a radius of about 60 mm. By tapering the tube-like body 2 towards its middle portion 26 the shape of the protector 1 is accommodated to the anatomy, thereby enhancing the wearing comfort.

FIG. 4 shows just another schematic side view of an embodiment of a protector 1 according to the invention, however, with the outer wall 14 having a more pronounced curvature. The radius which corresponds to the circle segment which forms the curvature of the outer wall 14 is in the range of 24 mm in this embodiment.

In FIG. 5 another embodiment of a protector 1 according to the invention is shown.

The only difference between the embodiment of FIG. 5 and that of FIGS. 1 to 3 lies in the fact that the first and second contact areas 16 and 18 of the slit 6 are not parallel in the ground state but are broadening when approaching the inner space 4. In such a manner at least in some cases the reversible release of the protector 1 from a toe strap can be facilitated, in particular if the elastomeric material of the tube-like body 2 is rather rigid.

In FIG. 6 another embodiment of protector 1 according to the present invention is shown. This protector device differs from that of FIGS. 1 to 3. essentially in that the slit 6 is no longer essentially parallel to the longitudinal axis, but is designed in the form of a bent curve from the first end to the second end 10. In this embodiment the first and second contact areas 16 and 18 in the ground state of the tube-like body 2 are preferably at least partially abutting each other. In a preferred embodiment the slit is off-set of the major transverse axis A at the first end and is also off-set of the major transverse axis A (and in particular also of the minor transverse axis B) at the second end 10 of the tube-like body 2. This embodiment encompasses at least two variants, one in which the slit does not cross the major transverse axis A from top end 8 to bottom end 10, and another in which the slit in fact crosses or intersects the major transverse axis A, the latter variant being less preferred.

Of course, any other curvature of the slit between the top end 8 and the bottom end 10 which departs from a straight line being essentially parallel to the longitudinal axis can be envisaged to be used with the protector of the present invention. For example, a zig-zag shaped slit is possible as well as a slit having elements which are essentially parallel to the longitudinal axis which are connected by slit elements which are not parallel to the longitudinal axis of the tube-like body. By using a slit which is not straight and not parallel to the longitudinal axis of the tube-like body the protector can sometimes even be prevented more effectively from escaping from the toe strap of footwear.

FIG. 7 shows another embodiment of a protector 1 according to the invention which essentially differs from the protector as shown in FIGS. 1 to 3 in that the contact areas 16 and 18 are provided with a male and female closure device 28 and 30, respectively. By pressing the first and second contact areas 16 and 18 towards each other the male and female closure devices 28, 30, which are shaped in the form of corresponding hooks, engage each other thereby closing the slit 6. In this manner a toe strap (not shown) is tightly enclosed in the inner space 4 during use. Advantageously, just by the pressure of adjacent toes exerted on the longitudinal side walls 22 and 24 of the tube-like body 2 a safe and reliable closure between male and female closure devices 28 and 30 is easily maintained. In the area of the back section 32 of the tube-like body where the first and second contact areas 16 and 18 are abutting each other the outer wall surface 14 is preferably continuously designed. In this longitudinal manner no outer step or rim is formed by a projecting element of a male or female closure device. Due to the elastic nature of the material used for the tube-like body 2 the closure provided by the male and female closure device can be easily manually opened in order to release a toe strap.

Although modifications and changes may be suggested by those skilled in the art, it is the intention of the applicant to embody within the patent warranted hereon all changes and modifications as reasonably and probably come within the scope of its contribution to the art. Especially the features disclosed in the foregoing description, in the claims and/or in the accompanying drawings may, both separately and in any combination thereof, be material for realizing the invention in diverse forms thereof.

### REFERENCE LIST

1 protector
2 tube or tube-like body
4 inner space
6 slit
8 first end
10 second end
12 inner wall
14 outer wall
16 first contact area of the slit
18 second contact area of the slit
20 front transverse section
22 first longitudinal section
24 second longitudinal section
26 middle portion of the tube or tube-like body
28 male closure device
30 female closure device
32 back section of the tube or tube-like body
34 wall/sleeve of the tube or tube-like body

We claim:

1-19. (canceled)

20. A protector for a toe strap of a footwear comprising the toe strap, the protector comprising, a tube-like body with a first end and an opposite second end along its longitudinal axis, the tube-like body forming a circumferential sleeve defining an inner space having a first opening at the first end of the protector and an opposite second opening at the second end of the protector for the at least partial inclusion of the toe strap within the inner space, wherein an inner wall of the tube-like body extends from the first end of the protector to the second end of the protector and wherein an outer wall extends from the first end of the protector to the second end of the protector, wherein the protector comprises a cross-section along at least a major portion of its longitudinal axis, wherein the tube-like body comprises a slit extending from the first end thereof to the second end thereof and from the outer wall thereof to the inner wall thereof, so that the tube-like body comprises adjacent longitudinal first and second contact areas which are facing each other and wherein the tube-like body comprises an elastomeric material.

21. The protector according to claim 20 wherein the cross-section at least along the major portion of the longitudinal axis of the protector is essentially circular.

22. The protector according to claim 20 wherein the cross-section at least along the major portion of the longitudinal axis of the protector is non-circular.

23. The protector according to claim 20 comprising a major transverse axis lying in the cross-section combining opposite portions of the outer wall of the tube-like body which are furthest apart from each other and a minor transverse axis being perpendicular to the major transverse axis and crossing the axis at half of its length, thereby generating four quadrants.

24. The protector according to claim 22 wherein the major transversal axis has a length in the range from about 5 mm to about 25 mm and the minor transverse axis has a length from about 3 mm to about 20 mm.

25. The protector according to claim 22 wherein the slit is located at an angle of at least more than 5° off of the major transverse axis and at an angle of at least more than 5° off of the minor transverse axis.

26. The protector according to claim 23 wherein the distance between the opposite portions of the outer wall which are lying on a transverse axis perpendicular to the longitudinal axis which is located at or adjacent to the first end and the second end is larger than the distance between opposite sections of the outer wall which are lying on a transverse axis perpendicular to the longitudinal axis which is located between the first end and the second end.

27. The protector according to claim 20 wherein the first contact area is provided with a male closure device and the second contact area is provided with a female closure device, wherein the male and female closure devices are adapted to releaseably engage each other.

28. The protector according to claim 20 wherein the elastomeric material is selected from the group consisting of a natural rubber, a synthetic rubber and a thermoplastic elastomer.

29. The protector according to claim 28 wherein the elastomeric material is a silicone rubber.

30. The protector according to claim wherein 20 the inner wall segment of the tube-like body at least partially is made from a polymeric material being more rigid than the material of the outer wall segment of the tube-like body.

31. Footwear comprising at least one protector for a toe strap according to claim 20.

32. Footwear according to claim 31 wherein the protector is an integral part of the insole or the strap of the footwear.
